(19) United States
(12) Patent Application Publication
(10) Pub. No.: US 2025/0042859 A1
(43) Pub. Date: Feb. 6, 2025

(54) METHOD FOR PREPARING 3,5-DINITROPYRAZOLE

(71) Applicants: ARIANEGROUP SAS, LES MUREAUX (FR); EURENCO, SORGUES (FR)

(72) Inventors: John EYMANN, VERT LE PETIT (FR); Nicolas VANDECANDELAERE, VERT LE PETIT (FR); Sébastien COMTE, VERT LE PETIT (FR); Angeline AUMELAS, VERT LE PETIT (FR)

(21) Appl. No.: 18/720,370

(22) PCT Filed: Dec. 15, 2022
(86) PCT No.: PCT/FR2022/052382

§ 371 Date: Jun. 14, 2024

(30) Foreign Application Priority Data
Dec. 17, 2021 (FR) FR2113765

(51) Int. Cl. C07D 231/38

(52) U.S. Cl. CPC C07D 231/38

(57) ABSTRACT

A method for preparing 3,5-dinitropyrazole, includes obtaining N-acetyl-3-nitropyrazole from pyrazole, and a sigmatropic rearrangement of the compound obtained.

### TECHNICAL FIELD OF THE INVENTION

The invention relates to the field of energetic molecules which can be used in the manufacture of pyrotechnic charges. More particularly, the invention relates to a method for preparing 3,4,5-trinitropyrazole precursors.

### STATE OF THE ART

Energetic molecules such as 3,4,5-trinitropyrazole (hereinafter, “3,4,5-TNP”) or derivatives of this compound (such as those described in EP-A-2 130 821) are suitable for the manufacture of pyrotechnic charges due to their interesting properties in terms of performance and sensitivity, while maintaining a high level of thermal stability, compatible with their use in the propulsion and explosives fields.

A route for synthesizing 3,4,5-TNP from 3,5-dinitropyrazole (hereinafter “3,5-DNP”) is described in application FR-A-2 917 409. Another method for preparing 3,4,5-TNP from 3,5-DNP is described in application CN105669557. Operating conditions for the sigmatropic rearrangement of N-nitropyrazole are described, for example, in J. Org. Chem. 1973, 38 (10), 1777-1782. These operating conditions (6 h at 160° C.) are considered limiting for development on an industrial scale. The result is poor efficiency and yield of the reaction to obtain 3,4,5-TNP from N-nitropyrazole. Application FR-A-3 085 377 describes a method for synthesizing 3,5-DNP from pyrazole; this method involves two sigmatropic rearrangement steps using microwave heating. However, the use of microwaves is a limiting factor for contemplating an industrial-scale development.

This observation led the inventors to look for solutions to improve the operating conditions for the synthesis of 3,5-DNP on an industrial scale.

### SUMMARY OF THE INVENTION

The inventors have demonstrated that it is possible to simplify the synthesis of 3,5-DNP by passing through a specific intermediate, namely N-acetyl-3-nitropyrazole. Thus, according to one aspect, the invention relates to a method for preparing 3,5-dinitropyrazole of formula (I):

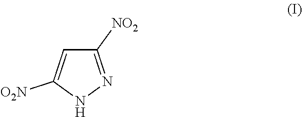 [SMILES: O=[N+]([O-])c1cc([N+](=O)[O-])[nH]n1]

which comprises:
a) the nitration of pyrazole using nitric acid and an excess of acetic anhydride relative to nitric acid;
b) the heat treatment, at a temperature of 160 to 240° C., of the mixture obtained in step a) to obtain a mixture containing a compound of formula (II):

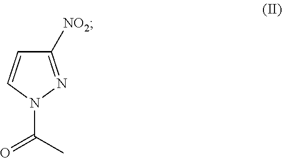

c) the hydrolysis of the mixture obtained in step b) to obtain a mixture containing a compound of formula (III):

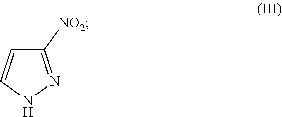

d) the nitration of the mixture obtained in step c) in the presence of acetic anhydride to obtain a mixture containing a compound of formula (IV):

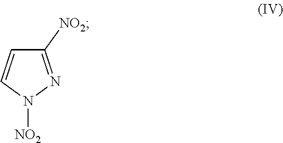 [SMILES: O=[N+]([O-])c1ccn([N+](=O)[O-])n1]

and
e) the heat treatment of the mixture obtained in step d).

### BRIEF DESCRIPTION OF THE DRAWINGS

FIGS. 1 and 2 illustrate the use of a continuous flow assembly to perform certain steps of the method according to the invention.

### DESCRIPTION OF THE INVENTION

According to a first aspect, the invention relates to a method for preparing 3,5-dinitropyrazole of formula (I):

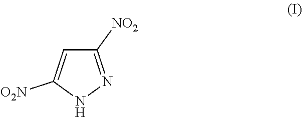 [SMILES: O=[N+]([O-])c1cc([N+](=O)[O-])[nH]n1]

which comprises:
a) the nitration of pyrazole using nitric acid and an excess of acetic anhydride relative to nitric acid;
b) the heat treatment, at a temperature of 160 to 240° C., of the mixture obtained in step a) to obtain a mixture containing a compound of formula (II):

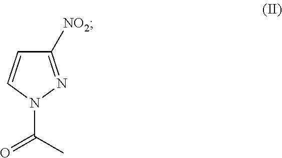

c) the hydrolysis of the mixture obtained in step b) to obtain a mixture containing a compound of formula (III):

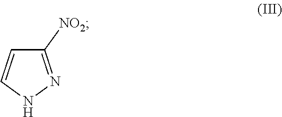

d) the nitration of the mixture obtained in step c) in the presence of acetic anhydride to obtain a mixture containing a compound of formula (IV):

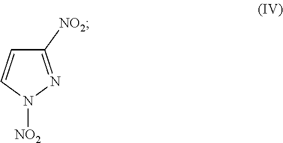 [SMILES: O=[N+]([O-])c1ccn([N+](=O)[O-])n1]

and
e) the heat treatment of the mixture obtained in step d).

Method step a) comprises the nitration of pyrazole. This step is carried out by reacting pyrazole with nitric acid in the presence of acetic anhydride. The presence of any other reagent and/or solvent is excluded during this nitration step.

In some embodiments, the nitration is carried out at a temperature of from about 0 to about 40° C., for example from about 10 to about 30° C. Advantageously, the nitration is carried out at a temperature in the range from 20 to 30° C.

In some embodiments, the duration of the nitration step is from about 5 minutes to about 1 hour, for example from about 5 minutes to about 50 minutes, or from about 10 minutes to about 40 minutes, in particular about 10 minutes, about 15 minutes, about 20 minutes, about 25 minutes or about 30 minutes.

The nitration is carried out with an excess of acetic anhydride compared to nitric acid, for example with about 2 to about 5 equivalents, in particular about 3 equivalents of acetic anhydride, per 1 equivalent of nitric acid.

The mixture obtained in step a) is used as such in the next step.

Method step b) comprises the heat treatment of the mixture obtained in step a) to obtain a mixture containing a compound of formula (II). As experimentally demonstrated (and illustrated in Example 1), this mixture always and mainly contains N-acetyl-3-nitropyrazole (compound of formula (II)), and may also contain N-acetylpyrazole. “Mainly” means that the mixture contains more than 50%, in particular more than 60%, of compounds of formula (II). This mixture is used as such for the next method step.

The heat treatment is carried out at a temperature of from about 160 to about 240° C., for example from about 180 to about 220° C., in particular of about 200° C. In the context of the present invention, the heat treatment is carried out using conventional heating means. For all intents and purposes, it will be clarified that microwave heating is not considered to be a conventional heating means. It is therefore excluded that the heat treatment is carried out using microwaves.

In some embodiments, the duration of the heat treatment is from about 5 minutes to about 1 hour, for example from about 5 minutes to about 45 minutes, in particular about 10 minutes, about 20 minutes, about 30 minutes or about 40 minutes.

In some embodiments, steps a) and b) are carried out in batch mode.

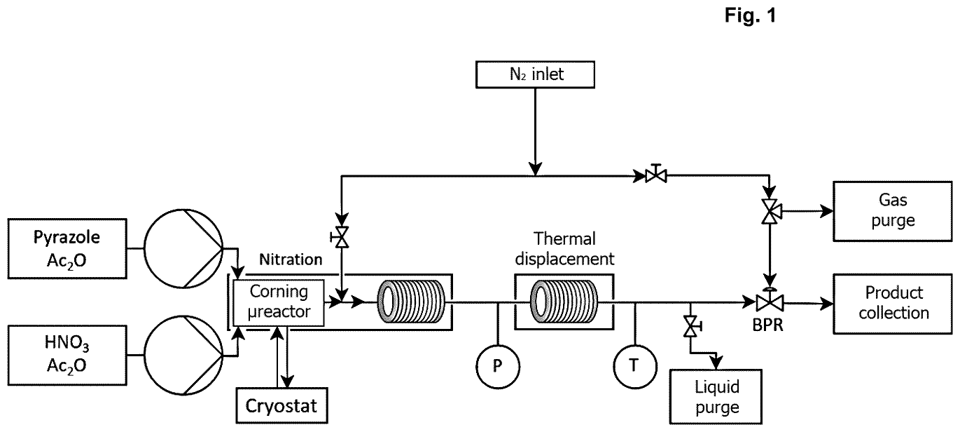

In some embodiments, steps a) and b) are performed in continuous flow. Continuous flow chemistry consists in carrying out syntheses in devices through which the flowing reaction medium passes, in which all reactions and physico-chemical transformations are carried out without isolating intermediates. FIG. 1 illustrates a flow chemistry set-up used in the following examples to couple steps a) and b).

Method step c) comprises the hydrolysis of the mixture obtained in step b) to obtain a mixture containing a compound of formula (III). This mixture is used as such for the following method step.

In some embodiments, the hydrolysis of the mixture is carried out at a temperature of from about 60 to about 120° C., for example from about 70 to about 110° C., in particular about 80° C.

In some embodiments, the duration of the hydrolysis step is from about 30 minutes to about 2 hours, for example from about 45 minutes to about 90 minutes, in particular about 1 hour.

In some embodiments, the hydrolysis is carried out with an excess of water relative to the pyrazole species present in the mixture, for example about 2 to about 5 equivalents, in particular about 3 equivalents of water, per 1 equivalent of pyrazole species.

Step d) comprises the nitration of the mixture obtained in step c) in the presence of acetic anhydride to obtain a mixture containing a compound of formula (IV). This mixture mainly contains 1,3-dinitropyrazole (compound of formula (IV)), as well as N-nitropyrazole, 3-nitropyrazole (III) and N-acetyl-3-nitropyrazole (II). It is used as such for the following method step.

The nitration step of the mixture obtained in step c) is advantageously carried out by reacting said mixture with nitric acid in the presence of acetic anhydride. In some embodiments, the nitration is carried out with an excess of acetic anhydride relative to nitric acid, for example about 2 to about 5 equivalents, in particular about 3 or about 4 equivalents of acetic anhydride, per 1 equivalent of nitric acid. The reaction between the mixture obtained in step c), nitric acid and acetic anhydride is advantageously carried out in the absence of any other reagent and/or solvent.

In some embodiments, the nitration is carried out at a temperature of from about 20 to about 40° C., for example from about 20 to about 30° C. Advantageously, the nitration is carried out at room temperature, which for the purposes of the present invention, is in the range of from about 20 to about 25° C.

In some embodiments, the duration of this second nitration step is from about 2 hours to about 24 hours, for example from about 5 hours to about 20 hours, or from about 10 hours to about 20 hours, in particular about 18 hours.

In some embodiments, steps c) and d) are carried out in continuous flow. In some embodiments, steps c) and d) are performed in batch mode and, advantageously, consecutively.

Step e) comprises the heat treatment of the mixture containing the compound of formula (IV) to obtain 3,5-DNP. The conditions specified for step b) also apply to step e), i.e. heat treatment by microwave is excluded.

In some embodiments, this second heat treatment step is carried out at a temperature of from about 160 to about 240° C., for example from about 180 to about 220° C., in particular about 200° C.

In some embodiments, the duration of this second heat treatment step is from about 5 minutes to about 1 hour, for example from about 5 minutes to about 50 minutes, in particular about 10 minutes, about 20 minutes, about 30 minutes or about 40 minutes.

In some embodiments, step e) is carried out in continuous flow.

According to another aspect, the invention relates to a method for preparing 3,4,5-trinitropyrazole which comprises:
a) the preparation of 3,5-dinitropyrazole by the method as defined above, and
b) the nitration of the resulting 3,5-dinitropyrazole.

The 3,5-DNP nitration step is carried out in a manner known to the skilled person, typically by reacting 3,5-DNP with fuming nitric acid in the presence of pure sulfuric acid and sulfuric oleum.

The method for preparing 3,5-DNP according to the invention has the following advantages:
a reduction in the number of synthesis steps;
a reduction in synthesis time and effluents;
the elimination of solvents, in particular toxic solvents such as bromobenzene, from the synthesis;
the use of a conventional heater for ease of installation;
a greater safety (thanks to the use of flow chemistry) and easier scale-up.

It will be noted in particular that steps b) to e) of the method according to the invention are carried out directly from the “raw” mixture obtained in the previous step, i.e. the mixture is used as such, without being purified, which ultimately reduces the total number of method steps, with an obvious economic advantage, particularly in terms of productivity on an industrial scale, and consequently improves the safety of the method taken as a whole. This is all the more true when the method steps are carried out in continuous flow, which enables mixing volumes to be reduced for the nitration steps, and temperature to be controlled during heat treatments.

The invention will be better understood with the aid of the following examples, given purely by way of illustration.

### EXAMPLES

### Equipment 1

The setup used for steps a) and b) of the 3,5-DNP preparation method is shown in FIG. 1. The equipment used is as follows:
2 piston pumps (FLOM Intelligent Pump UI-22);
3 Corning microreactors (1×LFSHH+2×LFR*H);
1 hot plate;
1 Back Pressure Regulator (BPR) (Zaiput BPR-10);
3 manual two-way valves;
1 manual three-way valve;
1 cryostat (Huber ministat 240);
3 thermocouples (1 in-line and two for the double envelope);
1 pressure sensor;
PFA tubes resistant to 200° C./10 bar;
Plastic connectors resistant to 18 bar;
2 scales;
1 nitrogen gas inlet.

The nitrogen gas inlet keeps the BPR setpoint at 10 bar, which is necessary to contain the gases formed during the thermal rearrangement. It can also be used to purge the system in case of emergency.

Solutions were prepared cold (0° C.) and then adjusted with volumetric flasks to obtain a precise concentration. Pyrazole solutions were injected at room temperature.

Conversely, acetonitric solutions (nitric acid+acetic anhydride (Ac₂O)) were kept at 0° C., even during injection, to avoid degradation. Solution mass flow rates are monitored using two scales, and solution density is used to determine volume flow rates.

### Operating Conditions 1 (for Steps a) and b))

Installation pressure: 10 bar;
Concentration of nitric acid in acetic anhydride (99% nitric acid): 317 g/L;
Concentration of pyrazole in acetic anhydride: 342 g/L;
Solution flow rates (equal for both solutions): 1 to 6 mL/min;
Jacket temperature of Corning microreactor: 28° C.;
Reaction loop temperature (nitration): RT;
Reaction loop temperature (thermal rearrangement): 200° C.;
Residence time (nitration): 15 minutes;
Residence time (thermal rearrangement): 10 minutes;
Dead time before collection: 1 hour.

### Equipment 2

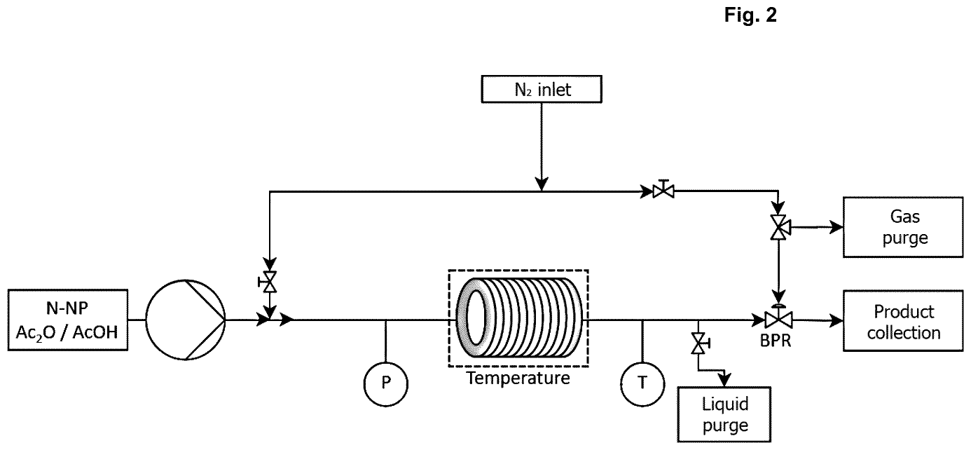

The set-up used for step e) of the 3,5-DNP preparation method is shown in FIG. 2. The equipment used is as follows:
1 piston pump (FLOM Intelligent Pump UI-22);
1 thermocouple (T);
1 pressure sensor (P);
3 manual two-way valves;
1 manual three-way valve;
1 back pressure regulator (Zaiput BPR-10);
PFA tubes resistant to 200° C./10 bar;
Plastic connectors resistant to 18 bar;
1 hot plate;
1 nitrogen gas inlet;
1 scale.
Operating conditions 2 (for step e))
Installation pressure: 10 bar;
Solution flow rates: 1 to 6 mL/min;
Reaction loop temperature (thermal rearrangement): 200° C.;
Residence time: 10 minutes;
Dead time before collection: 30 minutes.

### Example 1: N-acetyl-3-nitropyrazole

With reference to FIG. 1 and the equipment and operating conditions 1, 2.15 mL/min of a pyrazole solution in acetic anhydride (342 g of pyrazole per liter) and 2.15 mL/min of an acetonitric solution (317 g of fuming nitric acid per liter) were introduced in parallel into a first mixing microreactor. The reaction mixture was then reacted in a second microreactor for 15 min at 28° C. The reaction mixture was then transferred to a third microreactor for heat treatment (200° C. for 10 min). Following this heat treatment, a reaction mixture was obtained in the form of a clear orange liquid containing the title compound (508 g reaction mixture), which was used as such.

¹H NMR analysis of this medium revealed the presence of compound (II) at 67% and N-Acetylpyrazole at 33%.

¹H NMR (N-acetyl-3-nitropyrazole, CD₃NO₂, 400 MHZ, 21° C.): δ (ppm)=8.39 (d, 1H, NC(NO₂)CHCHN(Ac), ³J_{HH}=3.0 Hz), 7.11 (d, 1H, NC(NO₂)CHCHN(Ac), ³J_{HH}=2.9 Hz), 2.76 (s, 3H, CH₃).

¹H NMR (N-Acetylpyrazole, CD₃NO₂, 400 MHZ, 21° C.): δ (ppm)=8.26 (d, 1H, N(Ac)CHCHCHN, ³J_{HH}=2.8 Hz), 7.76 (s(b), 1H, N(Ac)CHCHCHN), 6.52 (dd, 1H, N(Ac)CHCHCHN, ³J_{HH}=2.8 Hz, ³J_{HH}=1.6 Hz), 2.65 (s, 3H, CH₃).

### Example 2: 3-nitropyrazole

The entire reaction mixture containing the N-acetyl-3-nitropyrazole obtained in Example 1, i.e. 508 g of solution, was placed in a 2 L round-bottomed flask. To this 65 mL of water were added portionwise (3.15 equivalents relative to the pyrazole species present in the mixture) and the mixture was heated for 1 h at 80° C. The reaction mixture obtained was a yellow liquid containing the title compound, which was used as such. Analysis by ¹H NMR revealed complete conversion of N-acetyl-3-nitropyrazole into 3-nitropyrazole.

¹H NMR (3-Nitropyrazole, CD₃NO₂, 400 MHZ, 21° C.): δ (ppm)=7.84 (d, 1H, NC(NO₂)CHCHNH, ³J_{HH}=2.6 Hz), 6.98 (d, 1H, NC(NO₂)CHCHNH, ³J_{HH}=2.6 Hz).

### Example 3: 1,3-dinitropyrazole

To the entire reaction mixture obtained in Example 2, acetic anhydride (430 mL) was added, followed by fuming nitric acid (47.6 mL, 1 equivalent relative to the pyrazole species present in the mixture), and the reaction mixture was left at room temperature for 18 hours. A new reaction mixture was obtained in the form of a clear yellow liquid containing the title compound, which was used as such. Proton NMR analysis revealed the presence of several pyrazole species and their relative proportions:
1,3-Dinitropyrazole (IV): 52%
N-Nitropyrazole: 32%
3-Nitropyrazole (III): 9%
N-acetyl-3-nitropyrazole (II): 7%

¹H NMR (1,3-Dinitropyrazole, CD₃NO₂, 400 MHZ, 21° C.): δ (ppm)=8.64 (d, 1H, NC(NO₂)CHCHN(NO₂), ³J_{HH}=3.1 Hz), 7.21 (d, 1H, NC(NO₂)CHCHN(NO₂), ³J_{HH}=3.1 Hz).

¹H NMR (N-Nitropyrazole, CD₃NO₂, 400 MHZ, 21° C.): δ (ppm)=8.47 (dd, 1H, N(NO₂)CHCHCHN, ³J_{HH}=3.0 Hz, 4J_{HH}=0.8 Hz), 7.71 (s(b), 1H, N(NO₂)CHCHCHN), 6.61 (dd, 1H, N(NO₂)CHCHCHN, ³J_{HH}=3.0 Hz, ³J_{HH}=1.7 Hz).

### Example 4: 3,5-dinitropyrazole

With reference to FIG. 2 and to the equipment and operating conditions 2, 4.3 mL/min of the 1,3-dinitropyrazole-containing reaction mixture obtained in Example 3 were introduced into a microreactor for heat treatment (200° C. for 10 min). Following this heat treatment, a reaction mixture was obtained in the form of a clear dark orange liquid containing the title compound, which was evaporated to dryness. The recovered solid was ground in 130 ml of dichloromethane. Filtration followed by washes with dichloromethane (2×50 mL) allowed the pure title compound to be isolated as a white solid (35.9 g, 27% yield vs. starting pyrazole).

### Characterization of the Final Product:

¹H NMR (3,5-Dinitropyrazole, Acetone d₆, 400 MHZ, 21° C.): (ppm)=7.82 (s, 1H, CH).

¹³C NMR (3,5-Dinitropyrazole, Acetone d₆, 100 MHZ, 21° C.): (ppm)=152.4 (CNO₂), 100.3 (CH).

DSC (40 μL aluminum crucible with pierced lid, 35 to 400° C., 5° C. min⁻¹): 170.4° C. (endo, −149.7 J·g⁻¹, melting), 240.3° C. (endo, −315.5 J·g⁻¹, evaporation).

1. A method for preparing 3,5-dinitropyrazole of formula (I): 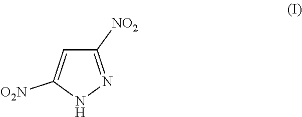 [SMILES: O=[N+]([O-])c1cc([N+](=O)[O-])[nH]n1]
which comprises:
a) carrying out a nitration of pyrazole using nitric acid and an excess of acetic anhydride relative to nitric acid;
b) heat-treating, at a temperature of about 160 to about 240° C., the mixture obtained in step a) to obtain a mixture containing a compound of formula (II): 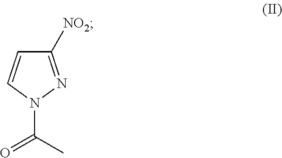
c) hydrolyzing the mixture obtained in step b) to obtain a mixture containing a compound of formula (III): 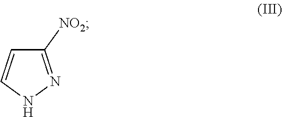
d) carrying out a nitration of the mixture obtained in step c) in the presence of acetic anhydride to obtain a mixture containing a compound of formula (IV): 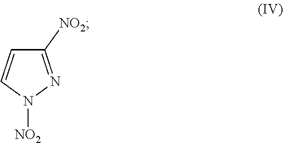 [SMILES: O=[N+]([O-])c1ccn([N+](=O)[O-])n1]
and
e) heat-treating the mixture obtained in step d).

2. The method of claim 1, wherein step a) is carried out at a temperature of about 0 to about 40° C.

3. The method of claim 2, wherein step a) is carried out for a period of about 5 min to about 1 h.

4. The method of claim 1, wherein step a) is carried out with about 2 to about 5 equivalents of acetic anhydride, per 1 equivalent of nitric acid.

5. The method of claim 1, wherein step b) is carried out for a period of about 5 min to about 1 h.

6. The method of claim 1, wherein step c) is carried out at a temperature of about 60 to about 120° C.

7. The method of claim 6, wherein step c) is carried out for a period of about 30 min to about 2 h.

8. The method of claim 6, in which step c) is carried out with an excess of water relative to the amount of pyrazole species present in the mixture obtained in step b).

9. The method of claim 8, wherein step c) is carried out with about 2 to about 5 equivalents of water per 1 equivalent of pyrazole species.

10. The method of claim 1, wherein step d) is carried out at a temperature of about 20 to about 40° C.

11. The method of claim 10, wherein step d) is carried out for a period of about 2 h to about 24 h.

12. The method of claim 10, wherein step d) is carried out using nitric acid and acetic anhydride, with an excess of acetic anhydride relative to nitric acid.

13. The method of claim 1, wherein step e) is carried out at a temperature of about 160 to about 240° C.

14. The method of claim 13, wherein step e) is carried out for a period of about 5 min to about 1 h.

15. The method of claim 1, wherein steps a), b) and e) are carried out in continuous flow.

16. The method of claim 1, wherein the nitration is carried out in steps a) and d) in the absence of any reagent and/or solvent other than acetic anhydride and nitric acid.

17. The method of claim 1, in which heat-treating in steps b) and e) is not carried out by microwaves.

18. A method for preparing 3,4,5-trinitropyrazole which comprises:
a) preparing 3,5-dinitropyrazole by the process as defined in claim 1, and
b) carrying out a nitration of the resulting 3,5-dinitropyrazole.

19. The method of claim 1, wherein step a) is carried out at a temperature of about 20 to about 30° C.

20. The method of claim 1, wherein steps c) and d) are carried out in continuous flow.
